(19) United States
(12) Patent Application Publication
(10) Pub. No.: US 2021/0205196 A1
(43) Pub. Date: Jul. 8, 2021

(54) PRESERVED COMPOSITIONS

(71) Applicant: Conopco, Inc., d/b/a UNILEVER, Englewood Cliffs, NJ (US)

(72) Inventors: Stuart CAMPBELL-LEE, Wirral (GB); Thomas Richard Pointon, Warrington (GB)

(21) Appl. No.: 17/056,661

(22) PCT Filed: May 20, 2019
(86) PCT No.: PCT/EP2019/062973

§ 371 Date: Nov. 18, 2020

(30) Foreign Application Priority Data
Jun. 4, 2018 (EP) 18175857.4

(51) Int. Cl. A61K 8/362; A61K 8/368; A61K 8/31; A61Q 5/02; A61K 8/41; A61K 8/46

(52) U.S. Cl. CPC A61K 8/362; A61K 8/368; A61K 8/31; A61Q 5/02; A61K 2800/5422; A61K 8/463; A61K 2800/524; A61K 2800/5428; A61K 2800/5424; A61K 8/416

(57) ABSTRACT

A composition comprising itaconic acid, a surfactant selected from anionic, nonionic, zwitterionic or amphoteric surfactants or mixtures thereof and at least 75 wt % water.

### FIELD OF THE INVENTION

The present invention relates to the area of preservation for personal care, in particular hair care compositions.

### BACKGROUND

In the personal care and cosmetic industry there is a constant need for preservation chemicals, especially preservation chemicals that are from natural sources, that are abundant and readily available.

A large number of antimicrobial active compounds are already employed in the personal care industry, but alternatives nevertheless continue to be sought. Not all antimicrobial agents have adequate preservation properties and thus the need for new preservation chemicals is particularly required. It is to be noted that the substances used in the personal care field must be:
toxicologically acceptable
readily tolerated by the skin
stable
largely and preferably completely odourless
inexpensive to prepare (i.e. employing standard processes and/or starting from standard precursors)
easy to formulate (i.e. preferably liquid) and should not be detrimental to the final product.

The present application has found a preservation chemical suitable for use with personal care compositions.

### DESCRIPTION OF THE INVENTION

The present invention relates to a composition comprising a short chain (C1 to C6), unsaturated, organic acid or salt thereof having at least two carboxyl groups and a surfactant selected from anionic, nonionic, zwitterionic or amphoteric surfactants or mixtures thereof.

The invention further relates to a method of preserving a composition according to any preceding claim by the use of; a short chain (C1 to C6), unsaturated, organic acid or salt thereof having at least two carboxyl groups.

### DETAILED DESCRIPTION OF THE INVENTION

Compositions of the invention comprise itaconic acid or salt thereof.

Preferably the total level of itaconic acid in the total composition is from 0.05 to 7 wt % by weight of the total composition, more preferably 0.1 to 5 wt % most preferably from 0.2 to 3 wt %, for example from 0.05 to 0.25 wt %.

Preferably the composition further comprises a cyclic, aromatic or acyclic organic compound and or terpene, more preferably the cyclic or acyclic compound comprises an aldehydes, ketones, amides, amine, alcohol, and ester group. Preferably the level of cyclic, aromatic or acyclic organic compound is from 0.05 to 3 wt % of the total composition.

In one preferred embodiment the cyclic or acyclic organic compound comprises an aromatic compound, preferably the aromatic compound is benzoic acid or salt thereof.

In a second preferred embodiment the composition further comprises a terpene and/or thymol, a particularly preferred terpenes is terpineol.

In a third embodiment the composition comprises a cyclohexane or derivative thereof.

Preferably the individual level of aromatic compound, benzoic acid or salt thereof or terpene is from 0.05 to 3 wt % of the total composition.

The compositions of the invention comprise at least 75 wt %, preferably at least 80 wt %, more preferably at least 85 wt % and most preferably at least 87 wt % of water, by weight of the total composition.

Preferably, the compositions of the invention are free from denatured ethanol, preferably denatured alcohol.

The composition according to the invention comprises a surfactant chosen from anionic, nonionic, zwitterionic or amphoteric surfactants or mixtures thereof.

Suitable anionic surfactants include the alkyl sulphates, alkyl ether sulphates, alkaryl sulphonates, alkanoyl isethionates, alkyl succinates, alkyl sulphosuccinates, N-alkoyl sarcosinates, alkyl phosphates, alkyl ether phosphates, alkyl ether carboxylates, and alpha-olefin sulphonates, especially their sodium, magnesium ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from one to 10 ethylene oxide or propylene oxide units per molecule, and preferably contain 2 to 3 ethylene oxide units per molecule.

Examples of suitable anionic surfactants include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauroyl isethionate and sodium N-lauryl sarcosinate. The most preferred anionic surfactants are sodium lauryl sulphate, triethanolamine lauryl sulphate, triethanolamine monolauryl phosphate, sodium lauryl ether sulphate 1EO, 2EO and 3EO, ammonium lauryl sulphate and ammonium lauryl ether sulphate 1EO, 2EO and 3EO.

Nonionic surfactants suitable for use in compositions of the invention may include condensation products of aliphatic (C8-C18) primary or secondary linear or branched chain alcohols or phenols with alkylene oxides, usually ethylene oxide and generally having from 6 to 30 ethylene oxide groups. Other suitable nonionics include mono- or di-alkyl alkanolamides, glycolipids preferably selected from the group of rhamnolipids and sophorolipids.

Examples include:

Amphoteric and zwitterionic surfactants suitable for use in compositions of the invention may include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates alkyl amidopropyl hydroxysultaines, acyl taurates and acyl glutamates, wherein the alkyl and acyl groups have from 8 to 19 carbon atoms. Examples include lauryl amine oxide, cocodimethyl sulphopropyl betaine and preferably lauryl betaine, cocamidopropyl betaine, coco mono- or di-ethanolamide, coco mono-isopropanolamide and sodium cocamphopropionate.

Generally, the surfactants are present in shampoo compositions of the invention in an amount of from 0.1 to 50%, preferably from 5 to 30%, more preferably from 10% to 25% by weight.

Preferably the weight ratio of anionic surfactant, preferably alkyl ether sulphate to unsaturated organic acid or salt thereof, preferably itaconic acid is from 200:1 to 2:1

Preferably the weight ratio of amphoteric or zwitterionic surfactant, preferably betaine to iunsaturated, organic acid or salt thereof, preferably itaconic acid is from 100:1 to 1:1.

Compositions of the invention are preferably personal care compositions, more preferably shampoo compositions.

The compositions of the invention may comprise silicone conditioning agent, preferably in the form of emulsified droplets for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is typically at least 10,000 cst at 25° C. the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in the shampoo compositions of the invention will typically have a D90 silicone droplet size in the composition of less than 30, preferably less than 20, more preferably less than 10 micron, ideally from 0.01 to 1 micron. Silicone emulsions having an average silicone droplet size (D50) of 0.15 micron are generally termed microemulsions. Silicone particle size may be measured by means of a laser light scattering technique, for example using a 2600D Particle Sizer from Malvern Instruments. Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation.

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”.

Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974. A preferred quaternary silicone polymer is K3474, ex Goldschmidt.

Also suitable are emulsions of amino functional silicone oils with nonionic and/or cationic surfactant. Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

The total amount of silicone is preferably from 0.01 to 10% wt of the total composition more preferably from 0.1 to 5 wt %, most preferably 0.5 to 3 wt % is a suitable level.

Preferably the weight ratio of silicone conditioning agent to short chain, unsaturated organic, acid is from 20:1 to 1:4.

Compositions of the invention may comprise further ingredients such as cationic deposition polymer preferably those selected from cationic cellulose and cationic guar derivatives. Other examples of ingredients found in compositions according to the invention comprise perfumes and pH adjusting agents.

The invention will now be illustrated by the following non-limiting examples.

### EXAMPLES

A shampoo base was prepared having the formulation according to Table 1.

TABLE 1
Shampoo formulation

| Ingredient | Percentage active |
|---|---|
| Water | 79.09 |
| Carbomer | 0.3 |
| Sodium laureth ether sulphate | 14 |
| Coco amidopropyl betaine | 1.6 |
| Guar hydroxypropyl trimethylammonium chloride C14 | 0.08 |
| Guar hydroxypropyl trimethylammonium chloride C17 | 0.03 |
| Dimethiconol | 1.5 |
| Ethylene glycol distearate | 1.25 |
| Titanium dioxide coated mica | 0.05 |
| Polyethylene glycol | 0.05 |
| Fragrance | 0.05 |
| Organic acid + pH adjustment | 2 |
| Total | 100 |

Itaconic acid, lactic acid and citric acid were added the conditioner composition at 0.5 wt % and 1 wt % of the total level of conditioner.

### Sample Preparation

Itaconic acid, lactic acid and citric acid were purchased from Sigma-Aldrich. Six samples of unpreserved conditioner were dosed with 0.5% and 1% (w/w) itaconic acid, citric acid and lactic acid separately. An unpreserved sample was kept as a control. Each dosed product was adjusted to pH 5 using 50% sodium hydroxide (Sigma-Aldrich).

### Challenge Test Method

A modified challenge test to that of the European Pharmacopoeia (Ph. Eur. Or EP) 5.1.3 criteria was performed on a subsample of unpreserved and each dosed product. The bacterial challenge test pool and inoculum level are summarised in Table 2. The microbial challenge pools were added to each sample container at a ratio of 1:100. The final concentration of inoculum in the product should be 5×10⁶ CFU/G of test product. Each product is mixed with a sterile spatula to ensure a homogenous distribution of the inocula throughout the product.

TABLE 2
Microbial Challenge test pool and Inoculum levels

| | | | Pool | In-product |
|---|---|---|---|---|
| | | | Inoculum | inoculum |
| Pool | Group | Microorganism | CFU/mL | CFU/G |
| 1 | Gram | Pseudomonas aeruginosa | 7.1 × 10⁸ | 5 × 10⁵⁻⁶ |
| | Negative | Burkholderia cepacia | CFU/mL | CFU/mL |
| | non- | Strenotrophomonas sp | | |
| | fermenting | | | |
| | bacteria | | | |
| 2 | Gram | Enterobacter cloacae | 5.2 × 10⁸ | 5 × 10⁵⁻⁶ |
| | Negative | Klebsiella sp | CFU/mL | CFU/mL |
| | fermenting | | | |
| | bacteria | | | |

Both the inoculum level and the level of microorganism within each sample was quantified using a Total Viable Count (TVC) pour plate method at 7, 14, 21 and 28 days. A 1:10 dilution was made with a subsample of each product, performed separately in peptone (0.1%)/tween 80 (2%) neutralising agent. A 1:10 and 1:100 dilution of each subsample was performed and pour plates produced at each dilution using tryptone soya agar (TSA). TSA plates were incubated at 28° C. for 48 hours and then examined for growth. Visible colonies were counted with the aid of a Quebec Colony Counter and recorded for analysis against the challenge test criteria. During the removal of a subsample of product for TVC, a reinoculation is performed at 7 and 14 days, reintroducing 5×10⁶ CFU/G of bacteria before mixing using a sterile spatula to homogenise the reinoculation.

TABLE 3
Microbial challenge test inoculation level

| Challenge | | Inoculum level (cfu/mL) | | |
|---|---|---|---|---|
| test pool | Microorganisms | Day 0 | Day 7 | Day 14 |
| Pool 1 | B. cepacia | 7.1E+08 | 7.8E+08 | 6.1E+08 |
| | P. aeruginosa | | | |
| | Strenotrophomonas sp | | | |
| Pool 2 | E. cloacae | 5.2E+08 | 4.1E+08 | 6.6E+08 |
| | Klebsiella sp. | | | |

TABLE 4
Microbial challenge test log kill

| | | | Microbial Log reduction | | | |
|---|---|---|---|---|---|---|
| | Sample | Pool | 7 days | 14 days | 21 days | 28 days |
| 1 | Unpreserved | 1 | >5.61 | 3.01 | <2.74 | >5.74 |
| | Shampoo | 2 | 3.94 | 4.61 | 4.72 | >5.80 |
| 2 | 0.5% Itaconic acid | 1 | >5.68 | >5.56 | >5.74 | >5.74 |
| | | 2 | >5.88 | >5.69 | >5.80 | >5.80 |
| 3 | 1.0% Itaconic acid | 1 | >5.61 | >5.56 | >5.74 | >5.74 |
| | | 2 | >5.88 | >5.69 | >5.80 | >5.80 |
| 4 | 0.5% Lactic acid | 1 | >5.61 | >5.56 | >5.74 | >5.74 |
| | | 2 | 3.93 | >5.69 | >5.80 | >5.80 |
| 5 | 1.0% Lactic acid | 1 | >5.61 | >5.56 | >5.74 | >5.74 |
| | | 2 | >5.88 | >5.69 | >5.80 | >5.80 |
| 6 | 0.5% Citric acid | 1 | >5.61 | 3.84 | >5.74 | >5.74 |
| | | 2 | 4.88 | >5.69 | >5.80 | >5.80 |
| 7 | 1.0% Citric acid | 1 | >5.61 | >5.56 | >5.74 | >5.74 |
| | | 2 | 4.97 | >5.69 | >5.80 | >5.80 |

The above table demonstrates the enhanced preservation properties of itaconic acid compared with other organic acids.

1. An aqueous composition comprising:
itaconic acid or salt thereof,
a surfactant selected from anionic, nonionic, zwitterionic or amphoteric surfactants or mixtures thereof, and
at least 75 wt % water.

2. The composition according to claim 1, which is free from denatured ethanol.

3. The composition according to claim 1, wherein the level of itaconic acid or salt thereof is from 0.05 to 7 wt % of the total composition.

4. The composition according to claim 1 further comprising an aromatic compound.

5. The composition according to claim 4, wherein the aromatic compound is benzoic acid or a salt thereof.

6. The composition according to claim 1 further comprising cyclohexane or a derivative thereof.

7. The composition according to claim 1 further comprising a terpene or thymol.

8. The composition according to claim 1 further comprising an aromatic compound, benzoic acid or salt thereof, cyclohexane, terpene and/or thymol, wherein the individual level of aromatic compound benzoic acid or salt thereof, cyclohexane, terpene or thymol-is from 0.05 to 3 wt % of the total composition.

9. The composition according to claim 1 comprising at least 80 wt % water of the total composition.

10. The composition according to claim 1 which is a shampoo.

11. The composition according claim 1, wherein the surfactant is anionic surfactant.

12. The composition according to claim 11, wherein the anionic surfactant comprises an alkyl ether sulphate.

13. The composition according to claim 11 in which the weight ratio of anionic surfactant to itaconic acid is from 200:1 to 2:1.

14. The composition according to claim 1, wherein the surfactant comprises an amphoteric or zwitterionic surfactant.

15. The composition according to claim 14, wherein the amphoteric or zwitterionic surfactant is a betaine.

16. The composition according to claim 14, wherein the weight ratio of amphoteric or zwitterionic surfactant to itaconic acid is from 100:1 to 1:1.

17. The composition according to claim 1 further comprising a silicone conditioning agent.

18. The composition according to claim 17, wherein the silicone conditioning agent is an amino functionalised silicone.

19. The composition according to claim 17, wherein the weight ratio of silicone conditioning agent to itaconic acid or salt thereof is from 20:1 to 1:4.

20. The method of preserving an aqueous composition as in claim 1 by the use of itaconic acid.
